(19) United States
(12) Patent Application Publication
(10) Pub. No.: US 2012/0004690 A1
(43) Pub. Date: Jan. 5, 2012

(54) SCREW FIXATION SYSTEM

(76) Inventor: Eduardo Gonzalez-Hernandez, Coconut Grove, FL (US)

(21) Appl. No.: 13/169,279

(22) Filed: Jun. 27, 2011

Related U.S. Application Data
(60) Provisional application No. 61/398,455, filed on Jun. 25, 2010.

(51) Int. Cl. A61B 17/86

(52) U.S. Cl. 606/305

(57) ABSTRACT

A screw fixation system serves to facilitate attachment of an orthopedic implant to a fractured bone to facilitate healing thereof. The screw fixation system includes the orthopedic implant, a bone screw, and a screw fixing element. The orthopedic implant includes an opening formed thereby. The screw fixing element has a body including an aperture extending between upper and lower surfaces thereof, and a channel adapted to receive a portion of the orthopedic implant adjacent the opening. When the portion of the orthopedic implant is received in the channel and the bone screw is inserted into the aperture, engagement of at least a portion of threads formed on a head of the bone screw and threads formed in the aperture expands the screw fixing element against the orthopedic implant to fix the bone screw and the orthopedic implant relative to one another.

The present application claims the benefit of provisional Application No. 61/398,455, filed Jun. 25, 2010, which is incorporated by reference herein.

### BACKGROUND OF THE INVENTION

1. Field of the Invention

The present invention is generally related to a system and method thereof for use in fixing a bone screw in relation to an orthopedic implant. More particularly, the present invention is related to a screw fixing element for securing a bone screw and an orthopedic implant in relation to one another. More specifically, the present invention is related to a screw fixing element for facilitating fixation of a bone screw at a predetermined angle relative to an orthopedic implant.

2. Description of the Prior Art

Surgical wires previously have been used by surgeons in repairing fractured bone. As such, the surgical wires have been used as orthopedic implants used in fixing fractured bone portions relative to one another to facilitate healing of the fractured bone. For example, given the inherent malleability thereof, the surgical wires can be wrapped around fractured bone portions to secure the bone portions relative to one another. Thus, when the surgical wires are wrapped around the fractured bone portions, the wires serve to maintain the position of the fracture bone portions to allow for healing thereof.

Additionally, fasteners have be used to secure engagement of the surgical wires to the fractured bone. For example, the surgical wires can be shaped to include portions for engaging the fasteners. To that end, the surgical wires can include loops to facilitate engagement with the fasteners. The loops can be sized to pass shafts of the fasteners therethrough, but prevent heads of the fasteners from passing therethrough. Thus, when the fasteners are passed through the loops and into the fractured bone, the heads engage portions of the surgical wires proximate the loops. In doing so, the heads effectively clamp the portions of the surgical wires proximate the loops to the fractured bone.

Given the inherent malleability of a surgical wire, however, the possibility exists that further deformation of the wire will cause disengagement thereof from a head of a fastener. Therefore, there is a need for a screw fixing element which engages the surgical wire to lock the fastener in position relative to the surgical wire, and in doing so, inhibit the wire from disengaging from the fastener due to further deformation thereof. Together, the surgical wire, the fastener, and the screw fixing element can be used in a screw fixation system to secure attachment of the surgical wire to bone.

### SUMMARY OF THE INVENTION

The present invention in a preferred embodiment contemplates a screw fixation system for use in an orthopedic procedure including an orthopedic implant, a bone screw, and a screw fixing element. The orthopedic implant includes a loop defining at least one opening. The bone screw has a trailing end, a leading end opposite the trailing end, a head portion proximate the trailing end, a shaft portion extending from the head portion to the leading end, a first set of threads formed on the head portion, and a second set of threads formed on the shaft portion. The second set of threads is adapted to engage bone. The screw fixing element has a body including an upper surface, an opposite lower surface, an aperture extending between the upper and lower surfaces, and a channel adapted to receive a portion of the orthopedic implant adjacent the at least one opening. The aperture has a central axis extending through the center of the aperture, and the aperture includes threads adapted to engage the first set of threads of the bone screw. The channel is proximate at least a portion of the perimeter of the body in a plane perpendicular to the central axis. When the portion of the orthopedic implant is received in the channel and the bone screw is inserted into the aperture, engagement of at least a portion of the first set of threads of the bone screw and the threads of the aperture expands the screw fixing element against the orthopedic implant to fix the bone screw and the orthopedic implant relative to one another.

The present invention in a further preferred embodiment contemplates a screw fixation system for use in an orthopedic procedure including an orthopedic implant, a bone screw, and a screw fixing element. The orthopedic implant is formed at least in part by a wire loop defining an opening. The bone screw has a trailing end, a leading end opposite the trailing end, a head portion proximate the trailing end, and a shaft portion extending from the head portion to the leading end. The head portion includes an exterior surface, and the shaft portion includes threads adapted to engage bone. The screw fixing element has a body including a channel adapted to receive a portion of the wire loop, and an aperture formed through the body. The aperture has a central axis extending through the center of the aperture, and the aperture includes an engagement surface adapted to engage the exterior surface of the head portion. Progressive engagement of the engagement surface and the exterior surface causes the screw fixing element to expand against the portion of the wire loop to fix the bone screw and the orthopedic implant relative to one another. Furthermore, when the bone screw and the orthopedic implant are fixed in position relative to one another, the screw fixing element covers more than half of a cross-sectional perimeter of the wire loop in a plane aligned with and extending through the central axis of the aperture.

The present invention in a still further preferred embodiment contemplates a method for facilitating repair of a fractured bone. The method includes the acts of providing an orthopedic implant formed at least in part by a wire loop defining an opening; providing a bone screw having a trailing end, a leading end opposite the trailing end, a head portion proximate the trailing end, and a shaft portion extending from the head portion to the leading end; providing a screw fixing element having a body including an upper surface, an opposite lower surface, an aperture extending between the upper and lower surfaces, and a channel adapted to receive a portion of the orthopedic implant adjacent the at least one opening, the aperture having a central axis extending through the center of the aperture, and the channel being provided proximate at least a portion of the perimeter of the body in a plane perpendicular to the central axis; introducing the screw fixing element into the opening of the wire loop, and inserting a portion of the wire loop into the channel of the screw fixing element; positioning the screw fixing element received in the opening of the wire loop adjacent a fractured bone; inserting the bone screw through the aperture of the screw fixing element and into the fractured bone; engaging threads on the head of the screw with threads in the aperture of the screw fixing element; tightening the bone screw against the screw fixing element to expand the screw fixing element against the portion of the wire loop received in the channel to fix the bone screw and the orthopedic implant relative to one another; and covering more than half of a cross-sectional perimeter of the wire loop in a plane aligned with and extending through the central axis of the aperture with the screw fixing element.

It is understood that both the foregoing general description and the following detailed description are exemplary and exemplary only, and are not restrictive of the invention as claimed.

### BRIEF DESCRIPTION OF THE DRAWINGS

The accompanying drawings, which are incorporated in and constitute a part of the specification, illustrate preferred embodiments of the invention. Together with the description, they serve to explain the objects, advantages and principles of the invention. In the drawings:

FIG. 1 is a perspective view of a screw fixation system according to the present invention depicting a bone screw partially inserted in an aperture of a screw fixing element received in a portion of an orthopedic implant;

FIG. 2 is a perspective view of the screw fixation system according to the present invention depicting the bone screw fully inserted in the aperture of the screw fixing element;

FIG. 3 is a perspective view of the portion of the orthopedic implant including an opening for receiving the screw fixing element;

FIG. 4 is a perspective view of the screw fixing element; and

FIG. 5 is a perspective view of the screw fixing element received in the opening of the orthopedic implant.

### DETAILED DESCRIPTION OF THE PREFERRED EMBODIMENT

The following description is intended to be representative only and not limiting, and many variations can be anticipated according to these teachings. Reference will now be made in detail to the preferred embodiments of this invention, examples of which are illustrated in the accompanying drawings.

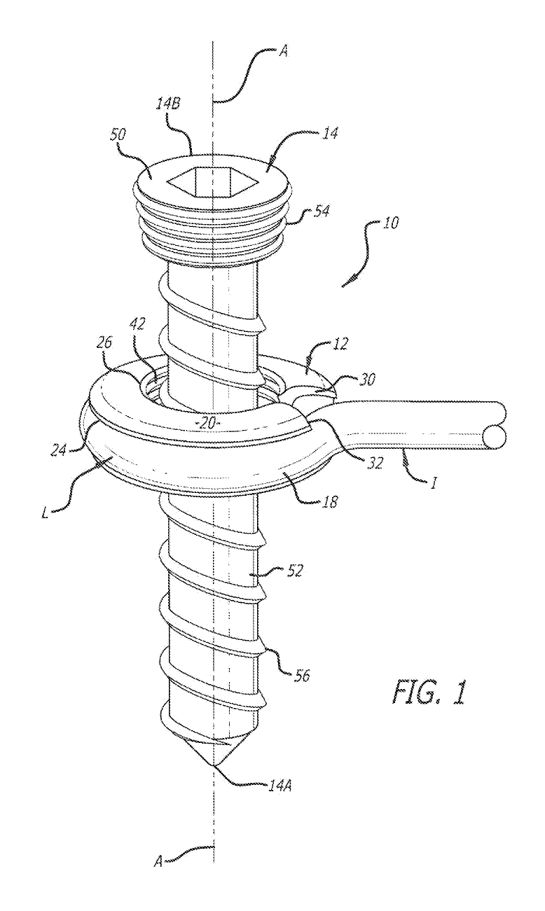

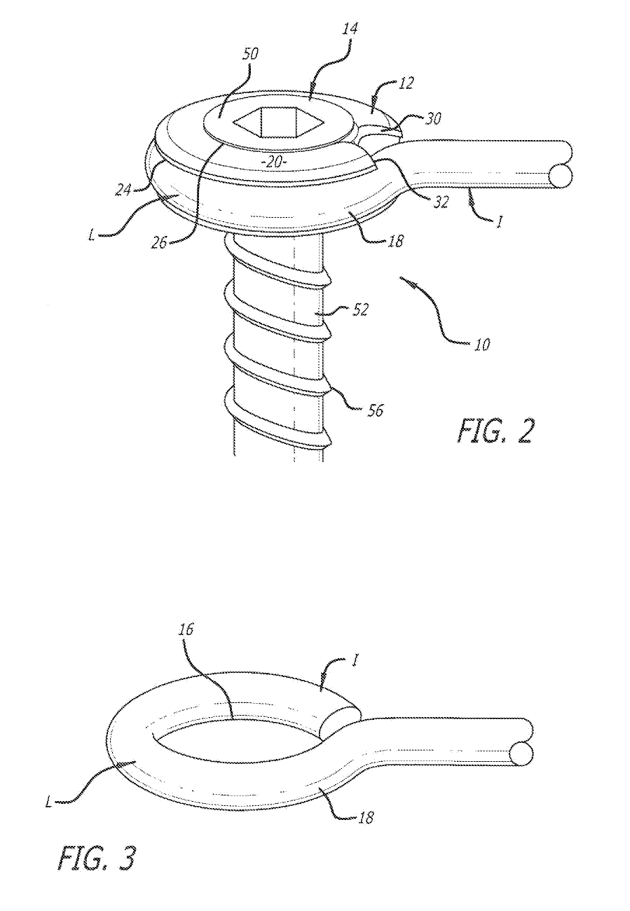

FIGS. 1 and 2 depict one preferred embodiment of the screw fixation system according to the present invention. The screw fixation system is generally indicated by the numeral 10 in FIGS. 1 and 2. As depicted in FIGS. 1 and 2, the screw fixation system 10 includes a screw fixing element 12, a fastener 14, and an orthopedic implant I. Furthermore, while fastener 14 depicted in FIGS. 1 and 2 is a bone screw including threads for engaging bone (and is hereinafter referred to as bone screw 14), fastener 14, for example, can instead include ratchets (not shown) for engaging bone.

Orthopedic implant I is provided for implantation into the human body, and, for example, can be used in repairing bone fractures. An end portion of orthopedic implant I is depicted in FIGS. 1-3 and 5, and is representative of other end portions of orthopedic implant I. Using screw fixing element 12 and bone screw 14 in combination therewith, orthopedic implant I can be used in joining fractured bone portions together.

Orthopedic implant I includes an opening 16 (FIG. 3), and a portion of orthopedic implant I adjacent the opening generally indicated by the numeral 18. Opening 16 is provided for receiving screw fixing element 12 and bone screw 14.

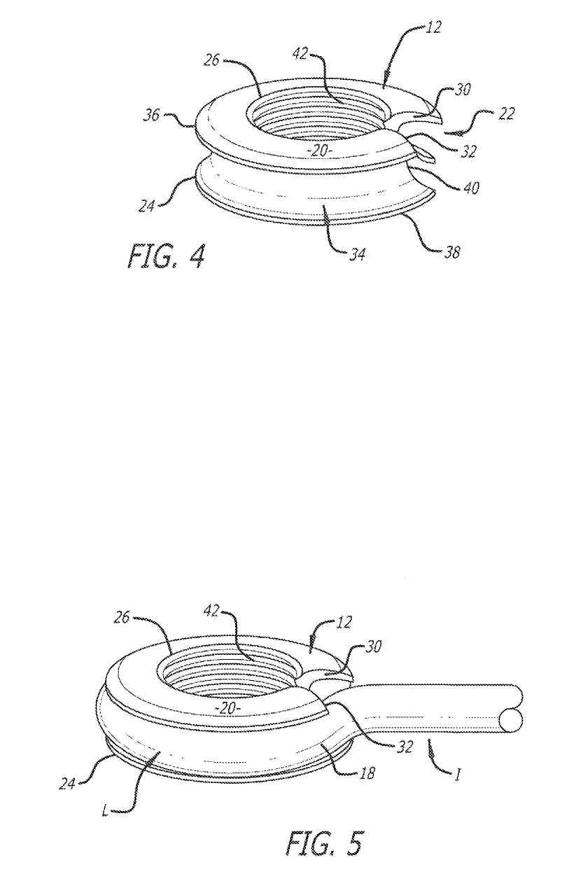

As depicted in FIGS. 1, 2, 4, and 5, screw fixing element 12 includes a body 20 shaped as a split toroid. That is, body 20 is shaped as a ring, but includes a gap 22 therein. Body 20 of screw fixing element 12 can be formed from metallic or polymeric materials allowing for the below-discussed contraction and expansion thereof.

The toroidal (or ring) shape of body 20 of screw fixing element 12 includes an upper surface, a lower surface, a perimeter 24, and an aperture 26. As depicted in FIGS. 1, 2, 4, and 5, perimeter 24 and aperture 26 have circular shapes. The shape of perimeter 24 of screw fixing element 12 is provided to correspond to the shape of opening 16. Thus, the circular shape of perimeter 24 is provided to correspond to the circular shape of opening 16 (FIG. 3). However, the shape of perimeter 24 and of body 20 is not limited to being circular and toroidal, respectively, and can be modified to accommodate the shape of opening 16 formed in orthopedic implant I.

Gap 22 affords contraction and expansion of the dimensions of screw fixing element 12 between a contracted position and a first expanded position. For example, a first end 30 and a second end 32 of screw fixing element 12 are spaced apart from one another by gap 22. First and second ends 30 and 32 can be pressed toward one another across gap 22 so that the spacing therebetween is decreased.

By pressing first and second ends 30 and 32 toward one another, screw fixing element 12 is also moved from the first expanded position to the contracted position, where outer perimeter 24 of screw fixing element 12 is smaller in the contracted position than in the first expanded position. Accordingly, when screw fixing element 12 is slightly larger than opening 16, the contraction afforded by gap 22 can permit screw fixing element 12 to be received therein.

Body 20 of screw fixing element 12 includes a channel 34 provided adjacent the perimeter thereof. Channel 34 is provided to receive portion 18 of orthopedic implant I adjacent opening 16.

Orthopedic implant I, as depicted in FIGS. 1, 2, 3, and 5 is formed by a wire. As such, opening 16 in orthopedic implant I is formed by a loop L of wire. Loop L does not have to be formed to have an uninterrupted perimeter to define opening 16 therein. Opening 16, for example, can also be formed as a hole in an orthopedic implant I formed as a plate. Whether formed by a wire or as a plate, portion 18 of orthopedic implant I adjacent opening 16 is received in channel 34 formed in screw fixing element 12.

Furthermore, channel 34 can be shaped to accommodate the shape of portion 18 of orthopedic implant I adjacent opening 16. Channel 34 includes an upper portion 36, a lower portion 38, and a connecting portion 40 connecting upper portion 36 and lower portion 38. Upper and lower portions 36 and 38 are opposed to one another and are spaced apart across the channel 34. Furthermore, depending on the cross-sectional shape of the wire, for example, upper portion 36, lower portion 38, and connecting portion 40 can be arranged differently. If the wire has a circular cross section (FIGS. 1-3 and 5), upper portion 36, lower portion 38, and connecting portion 40 can be arcuate, and can be arranged to approximate a portion of a circle in cross section; and if the wire has a square cross section by way of example, upper portion 36, lower portion 38, and connecting portion 40 can be straight, and can be arranged to approximate a portion of a square in cross section.

Aperture 26 formed through body 20 is provided for engaging bone screw 14. To that end, aperture 26 can include threads 42 or be threadless. Whether including threads 42 or being threadless, aperture 26 can be configured to have a complementary shape to facilitate engagement with bone screw 14. For example, aperture 26 can have a cylindrical or frusto-conical shape. Furthermore, aperture 26 also can be angled to facilitate fixation of bone screw 14 relative to orthopedic implant I. For example, aperture 26 can be angled so that the axis thereof is parallel to the axis of opening 16. Furthermore, aperture 26 can be angled so that the axis thereof is transverse to the axis of opening 16. Thus, by providing the axis of aperture 26 at a particular angle, the fixation of bone screw 14 relative to orthopedic implant I can be provided at a predetermined angle.

As depicted in FIG. 1, bone screw 14 includes a leading end 14A, a trailing end 14B, and an mid-longitudinal axis A extending through leading and trailing ends 14A and 14B. Bone screw 14 includes a head portion 50 proximate trailing end 14B and a shaft portion 52 extending downwardly from head portion 50 to leading end 14A. Head portion 50 can include threads 54 or be threadless. Furthermore, shaft portion 52 includes threads 56 formed thereon for engaging bone.

The frusto-conical shape of head portion 50 can complement a cylindrical or frusto-conical shape of aperture 26. As such, the interaction between the complementary shapes of aperture 26 and frusto-conical portion 50 can serve to expand body 20 to a second expanded position. Outer perimeter 24 can be larger in the second expanded position than in the first expanded position, and, as discussed below, when in the second expanded position, screw fixing element 12 can be locked in position relative to orthopedic implant I. Furthermore, if head portion 50 includes threads 54, threads 54 can matingly engage at least a portion of threads 42 of aperture 26.

During use of screw fixation system 10, screw fixing element 12 is initially positioned with respect to orthopedic implant I. As such, first and second ends 30 and 32 are pressed together to move screw fixing element 12 from the first expanded position to the contracted Position. Screw fixing element 12 is then positioned in opening 16. Once positioned in opening 16, screw fixing element 12 is permitted to move from the contracted position to the first expanded position or to an expanded position more or less than the first expanded position. In doing so, portion 18 of orthopedic implant I adjacent opening 16 is received in channel 34 formed in body 20 of screw fixing element 12.

Once screw fixing element 12 is positioned with respect to orthopedic implant I, bone screw 14 can be inserted in opening 26 (FIG. 2). In doing so, head portion 50 is contacted against aperture 26, and the complementary shapes of aperture 26 and head portion 50 interact with one another. If aperture 26 and head portion 50 are threaded, at least a portion of threads 54 of head portion 50 are then matingly engaged with at least a portion of threads 42 of aperture 26. Due to the frusto-conical shape of head portion 50, interaction between aperture 26 and head portion 50 force body 20 of screw fixing element 12 to expand against portion 18 of orthopedic implant I adjacent opening 16. In doing so, connecting portion 40 of channel 34 is forced outwards against portion 18 of orthopedic implant I adjacent opening 16 to lock screw fixing element 12 in position relative to orthopedic.

Other embodiments of the invention will be apparent to those skilled in the art from consideration of the specification and practice of the invention disclosed herein. Accordingly, it is intended that the specification and examples be considered as exemplary only, with a true scope and spirit of the invention being indicated by the following claims.

What is claimed is:

1. A screw fixation system for use in an orthopedic procedure, the screw fixation system comprising:
an orthopedic implant including a loop defining at least one opening;
a bone screw having a trailing end, a leading end opposite said trailing end, a head portion proximate said trailing end, a shaft portion extending from said head portion to said leading end, a first set of threads formed on said head portion, and a second set of threads formed on said shaft portion, said second set of threads being adapted to engage bone; and
a screw fixing element having a body including an upper surface, an opposite lower surface, an aperture extending between said upper and lower surfaces, and a channel adapted to receive a portion of said orthopedic implant adjacent said at least one opening, said aperture having a central axis extending through the center of said aperture, and said aperture including threads adapted to engage said first set of threads of said bone screw, and said channel being provided proximate at least a portion of the perimeter of said body in a plane perpendicular to the central axis, wherein, when said portion of said orthopedic implant is received in said channel and said bone screw is inserted into said aperture, engagement of at least a portion of said first set of threads of said bone screw and said threads of said aperture expands said screw fixing element against said orthopedic implant to fix said bone screw and said orthopedic implant relative to one another.

2. The screw fixation system of claim 1, wherein said body is at least in part arcuate in the plane perpendicular to the central axis of said aperture.

3. The screw fixation system of claim 1, wherein said channel of said screw fixing element includes an upper portion and a lower portion, said upper and lower portions being spaced apart from one another across said channel.

4. The screw fixation system of claim 3, wherein said upper and lower portions are connected to one another by a connecting portion, said connecting portion being provided proximate said aperture through said body.

5. The screw fixation system of claim 3, wherein said upper and lower portions of said channel each are at least in part arcuate in a plane aligned with and extending through the central axis of said aperture.

6. The screw fixation system of claim 3, wherein said channel has a generally C-shaped cross-section in the plane extending through the central axis of said aperture.

7. The screw fixation system of claim 3, wherein said upper and lower portions are moveable with respect to one another to afford passage of said portion of said orthopedic implant into said channel.

8. The screw fixation system of claim 7, wherein said upper and lower portions expand to a first position to pass said portion of said orthopedic implant into said channel, and said upper and lower portions contract to a second position to retain said portion of said orthopedic implant in said channel.

9. The screw fixation system of claim 1, wherein said body of said screw fixing element includes a first end, a second end, and a gap spacing said first and second ends apart from one another.

10. The screw fixation system of claim 9, wherein said gap between said first and second ends is adapted to decrease in size when said screw fixing element is contracted in the plane perpendicular to the central axis, and adapted to increase when said screw fixing element is expanded in the plane perpendicular to the central axis.

11. The screw fixation system of claim 1, wherein said body of said screw fixing element includes a first end and a second end, said first and second ends being movable with respect to one another between an expanded first position and a contracted second position, wherein, when in said contracted second position, said screw fixing element can be received within said aperture of said orthopedic implant.

12. A screw fixation system for use in an orthopedic procedure, the screw fixation system comprising:
an orthopedic implant formed at least in part by a wire loop defining an opening;
a bone screw having a trailing end, a leading end opposite said trailing end, a head portion proximate said trailing end, and a shaft portion extending from said head portion to said leading end, said head portion including an exterior surface, and said shaft portion including threads adapted to engage bone; and
a screw fixing element having a body including a channel adapted to receive a portion of said wire loop, and an aperture formed through said body, said aperture having a central axis extending through the center of said aperture, and said aperture including an engagement surface adapted to engage said exterior surface of said head portion, wherein progressive engagement of said engagement surface and said exterior surface causes said screw fixing element to expand against said portion of said wire loop to fix said bone screw and said orthopedic implant relative to one another, and, when said bone screw and said orthopedic implant are fixed in position relative to one another, said screw fixing element covers more than half of a cross-sectional perimeter of said wire loop in a plane aligned with and extending through the central axis of said aperture.

13. The screw fixation system of claim 12, wherein said channel is provided proximate more than half of the perimeter of said body in a plane perpendicular to the central axis of said aperture.

14. The screw fixation system of claim 13, wherein said channel of said screw fixing element includes an upper portion and a lower portion spaced apart from one another across said channel, said upper and lower portions being adapted to expand to a first position to pass said portion of said wire loop into said channel, and said upper and lower portions being adapted contact a second position to retain said portion of said orthopedic implant in said channel.

15. The screw fixation system of claim 12, further comprising threads provided on at least a portion of said exterior surface of said head portion, and threads provided on a least a portion of said engagement surface of said aperture of said screw fixing element, wherein said threads of said exterior surface and said engagement surface are adapted to engage one another.

16. The screw fixation system of claim 15, wherein engagement of said threads of said exterior surface and said engagement surface forces expansion of said screw fixing element.

17. The screw fixation system of claim 12, wherein said body of said screw fixing element includes an upper surface, an opposite lower surface, and said aperture extends between said upper and lower surfaces, and, when said bone screw and said orthopedic implant are fixed in position relative to one another, said trailing end of said bone screw is substantially flush with said upper surface of said screw fixing element.

18. A screw fixation system for use in an orthopedic procedure, the screw fixation system comprising:
an orthopedic implant including a loop defining at least one opening;
a bone screw having a trailing end, a leading end opposite said trailing end, a head portion proximate said trailing end, a shaft portion extending from said head portion to said leading end, a first set of threads formed on said head portion, and a second set of threads formed on said shaft portion, said second set of threads being adapted to engage bone; and
a screw fixing element having a body including an upper surface, an opposite lower surface, an aperture extending between said upper and lower surfaces, and a channel adapted to receive a portion of said orthopedic implant adjacent said at least one opening, said aperture having a central axis extending through the center of said aperture, and said aperture including threads adapted to engage said first set of threads of said bone screw, said body being at least in part arcuate in a plane perpendicular to the central axis of said aperture, said body including a first end, a second end, and a gap spacing said first and second ends apart from one another, and said channel being provided proximate at least a portion of the perimeter of said body in the plane perpendicular to the central axis, said channel having a generally C-shaped cross-section in a plane aligned with and extending through the central axis of said aperture, and said channel including an upper portion and a lower portion being spaced apart from one another across said channel, said upper and lower portions being connected to one another by a connecting portion, said connecting portion being provided proximate said aperture through said body, said upper and lower portions each being at least in part arcuate in a plane aligned with and extending through the central axis of said aperture, said upper and lower portions being expandable to a first position to pass said portion of said orthopedic implant into said channel, and said upper and lower portions being contractable to a second position to retain said portion of said orthopedic implant in said channel, wherein said gap between said first and second ends is adapted to decrease in size when said screw fixing element is contracted in the plane perpendicular to the central axis, and adapted to increase when said screw fixing element is expanded in the plane perpendicular to the central axis, and wherein, when said portion of said orthopedic implant is received in said channel and said bone screw is inserted into said aperture, engagement of at least a portion of said first set of threads of said bone screw and said threads of said aperture expands said screw fixing element against said orthopedic implant to fix said bone screw and said orthopedic implant relative to one another.

19. A method for facilitating repair of a fractured bone, the method comprising:
providing an orthopedic implant formed at least in part by a wire loop defining an opening;
providing a bone screw having a trailing end, a leading end opposite the trailing end, a head portion proximate the trailing end, and a shaft portion extending from the head portion to the leading end;
providing a screw fixing element having a body including an upper surface, an opposite lower surface, an aperture extending between the upper and lower surfaces, and a channel adapted to receive a portion of the orthopedic implant adjacent the at least one opening, the aperture having a central axis extending through the center of the aperture, and the channel being provided proximate at least a portion of the perimeter of the body in a plane perpendicular to the central axis;
introducing the screw fixing element into the opening of the wire loop, and inserting a portion of the wire loop into the channel of the screw fixing element;
positioning the screw fixing element received in the opening of the wire loop adjacent a fractured bone;
inserting the bone screw through the aperture of the screw fixing element and into the fractured bone;
engaging threads on the head of the screw with threads in the aperture of the screw fixing element;
tightening the bone screw against the screw fixing element to expand the screw fixing element against the portion of the wire loop received in the channel to fix the bone screw and the orthopedic implant relative to one another; and
covering more than half of a cross-sectional perimeter of the wire loop in a plane aligned with and extending through the central axis of the aperture with the screw fixing element.
